(19) United States
(12) Patent Application Publication
(10) Pub. No.: US 2006/0140993 A1
(43) Pub. Date: Jun. 29, 2006

(54) Hair and Scalp Ointment

(76) Inventor: Donna Jean Russau Johnson, Durham, NC (US)

Correspondence Address: DONNA JEAN RUSSAU JOHNSON, 605 LEADER LANE, DURHAM, NC, 27703, US

(21) Appl. No.: 11/242,965

(22) Filed: Oct. 4, 2005

Related U.S. Application Data
(60) Provisional application No. 60/616,667, filed on Oct. 7, 2004.

(51) Int. Cl. A61K 8/18

(52) U.S. Cl. 424/401

(57) ABSTRACT

Abies Balsamea Resin, Beeswax, Carmel Color, Cetyl Alcohol, Disodium Edta, DMDH Hydantoin, Fragrance, Gelatin/Keratin Amino Acids/Lysine Hydroxypropyltrimonium Chloride, Glycerin, Stearate, Propylene Glyco and Imidazolidinyl Urean and Methyl Paraben and Propyl Paraben, Hydrolyzed Collagen, Microcrystalline Wax, Octoxynol-9, Panthenol (Pro Vitamin B5), Petrolatum, Propylene, Sodium Salt of Carbomer 940, Vitamin A, Vitamin C, Vitamin E, Water, Yellow 5, Yellow 6, Yellow 10. All of the above ingredients are cooked and mixed together.

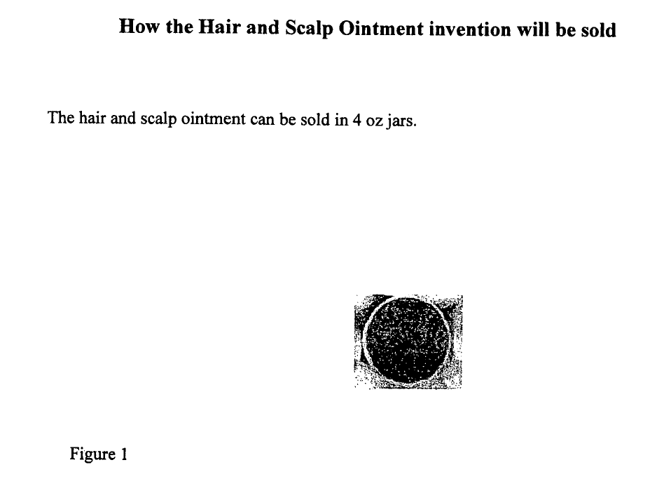

### SPECIFICATIONS

In reference to the provisional patent pending (application No. 60/616,667) for the hair and scalp ointment that I have invented, I herewith describe the product for which I am applying for a non-provisional application; it is as follows:

The hair and scalp ointment is a non-greasy cream made from a variety of ingredients (please see abstract/ingredients) blended together. The hair product helps keep the scalp and hair from drying, while maintaining moisture and promoting shine of the hair for all types of hair types, especially coarse textures.

1. I, Donna Jean Russau Johnson, the inventor of the hair and scalp ointment, claim that my hair ointment will help keep the scalp and hair from drying out after parting and applying it liberally to the hair in sections. My hair and scalp ointment will help maintain moisture and promote shine of the hair for natural hair styles such as braids, twists, locks, afro and more. My hair and scalp ointment also offers versatility to manage spikes, finger waves, the slick look, wispy designs along with other straight hair styles. Ultimately, my hair and scalp ointment can supply hair care for the vast majority of men and women of all heritages.
